(19) United States
(12) Patent Application Publication
(10) Pub. No.: US 2025/0042833 A1
(43) Pub. Date: Feb. 6, 2025

(54) PRODUCTION METHOD FOR AROMATIC HYDROXYCARBOXYLIC ACID CRYSTAL

(71) Applicant: KAO CORPORATION, Tokyo (JP)

(72) Inventors: Sawa MIZOBATA MIYAKE, Wakayama-shi, Wakayama (JP); Yutaka IRIE, Wakayama-shi, Wakayama (JP); Masahiro NOBA, Wakayama-shi, Wakayama (JP)

(73) Assignee: KAO CORPORATION, Tokyo (JP)

(21) Appl. No.: 18/716,977

(22) PCT Filed: Dec. 2, 2022
(86) PCT No.: PCT/JP2022/044608

§ 371 Date: Jun. 6, 2024

(30) Foreign Application Priority Data
Dec. 6, 2021 (JP) 2021-197546

(51) Int. Cl. C07C 51/43; C07C 51/347

(52) U.S. Cl. CPC C07C 51/43; C07C 51/347; C07B 2200/13

(57) ABSTRACT

Provided is a method for producing a high-purity aromatic hydroxycarboxylic acid crystal. A method for producing an aromatic hydroxycarboxylic acid crystal, the method including a step of precipitating an aromatic hydroxycarboxylic acid crystal from a solution including aromatic hydroxycarboxylic acid and at least one polymer molecule selected from the group consisting of an anionic polymer molecule, a non-ionic polymer molecule and a cationic polymer molecule, in which a content of the polymer molecule in the solution is 0.0008 mass % or more and 16 mass % or less.

### FIELD OF THE INVENTION

The present invention relates to a method for producing an aromatic hydroxycarboxylic acid crystal.

### BACKGROUND OF THE INVENTION

Aromatic hydroxycarboxylic acid is a collective term for an aromatic compound having both a hydroxy group and a carboxy group in a molecule. Representative examples include salicylic acid, 4-hydroxybenzoic acid, gallic acid (another name: 3,4,5-trihydroxybenzoic acid), and protocatechuic acid. Aromatic hydroxycarboxylic acid is an important compound as a raw material or an intermediate for electronic materials, liquid crystal materials, pharmaceutical products, foods, cosmetics, and the like.

Aromatic hydroxycarboxylic acid such as salicylic acid or 4-hydroxybenzoic acid has been industrially produced for years by an organic chemical synthesis method known as Kolbe-Schmitt reaction. Gallic acid is produced by hydrolysis of tannin extracted from insect gall of plant Chinese sumac, Rhus javanica L., with alkali, acid, or an enzyme.

Meanwhile, there has been recently developed a process for producing aromatic hydroxycarboxylic acid with microorganisms, because of, for example, the problems of environmental impact and safety. For example, a method for producing gallic acid from glucose by Escherichia coli with a mutant enzyme has been reported (Patent Literature 1). Patent Literature 1 has reported to produce a gallic acid crystal obtained a fermentation culture solution obtained by Escherichia coli with glucose as a raw material, by poor solvent crystallization.

Both production methods of an organic chemical synthesis method and fermentative production by microorganisms are required a step of purifying an aromatic hydroxycarboxylic acid crystal as a desired product, from a by-product. However, aromatic hydroxycarboxylic acid is likely turned into a needle crystal, and consequently is highly formed into a fine crystal. Therefore, crystal separation efficiency is reduced, and thus a mother liquid likely remains in a crystal. Moreover, washing failure, or aggregation during drying likely occurs. Consequently, it results in a reduction in crystal purity.

There has been conventionally reported, as a crystallization method for obtaining a high-purity objective crystal, for example, it includes allowing a surfactant and/or an alcohol to exist in an amino acid solution to change the crystal form, during crystallization of amino acid (Patent Literature 2). Patent Literature 2 discloses that due to an addition of a surfactant and/or an alcohol, a crystal grows to a larger size, to facilitate a solid-liquid separation operation, and decreasing an attached amount of a mother liquid, it results in obtaining a high-purity amino acid crystal. Patent Literature 2 fails to report any crystallization of an aromatic hydroxycarboxylic acid crystal.
(Patent Literature 1) US B 6472190
(Patent Literature 2) WO 90/09372

### SUMMARY OF THE INVENTION

The present invention provides a method for producing an aromatic hydroxycarboxylic acid crystal, the method including a step of precipitating an aromatic hydroxycarboxylic acid crystal from a solution including aromatic hydroxycarboxylic acid and at least one polymer molecule selected from the group consisting of an anionic polymer molecule, a non-ionic polymer molecule and a cationic polymer molecule, wherein a content of the polymer molecule in the solution is 0.0008 mass % or more and 16 mass % or less.

### DETAILED DESCRIPTION OF THE INVENTION

The present invention relates to provision of a method for producing a high-purity aromatic hydroxycarboxylic acid crystal.

The present inventor found that if aromatic hydroxycarboxylic acid is precipitated in the presence of an anionic polymer molecule, a non-ionic polymer molecule or a cationic polymer molecule, it results in an increase in crystal size and thus obtaining a high-purity aromatic hydroxycarboxylic acid crystal.

According to the method of the present invention, it provides an aromatic hydroxycarboxylic acid crystal with large crystal size. Thus, it leads to an advantage that crystal separation efficiency is improved and a high-purity aromatic hydroxycarboxylic acid crystal can be obtained.

### [Method for Producing Aromatic Hydroxycarboxylic Acid Crystal]

The method for producing an aromatic hydroxycarboxylic acid crystal of the present invention includes a step of precipitating an aromatic hydroxycarboxylic acid crystal from a solution including aromatic hydroxycarboxylic acid and at least one polymer molecule selected from the group consisting of an anionic polymer molecule, a non-ionic polymer molecule and a cationic polymer molecule. Hereinafter, the “solution including aromatic hydroxycarboxylic acid and at least one polymer molecule selected from the group consisting of an anionic polymer molecule, a non-ionic polymer molecule and a cationic polymer molecule” is herein also referred to as “the solution”.

The at least one polymer molecule selected from the group consisting of an anionic polymer molecule, a non-ionic polymer molecule and a cationic polymer molecule is needed to be present when the aromatic hydroxycarboxylic acid crystal is precipitated. The timing of addition of the polymer molecule to the solution including aromatic hydroxycarboxylic acid is not particularly limited.

### (Aromatic Hydroxycarboxylic Acid)

Examples of the aromatic hydroxycarboxylic acid herein include salicylic acid, γ-resorcinol acid, hydroxybenzoic acid, aminohydroxybenzoic acid, gallic acid, protocatechuic acid, or a compound obtained by substituting at least some hydrogen on an aromatic ring of such aromatic hydroxycarboxylic acid, with a substituent. Examples of the substituent include an alkyl group, an amino group, and an aryl group. The “aromatic hydroxycarboxylic acid” does not herein encompass any aromatic hydroxycarboxylic acid salt such as an alkali metal salt of the aromatic hydroxycarboxylic acid.

The aromatic hydroxycarboxylic acid may contain a plurality of carboxy groups, and preferably contains one carboxy group. The aromatic hydroxycarboxylic acid preferably contains from one to three hydroxy groups. In particular, the aromatic hydroxycarboxylic acid is preferably hydroxybenzoic acid, aminohydroxybenzoic acid, gallic acid, or protocatechuic acid, more preferably gallic acid or protocatechuic acid because the effects of the present invention are easily enjoyed.

The aromatic hydroxycarboxylic acid can be obtained by an organic chemical synthesis method, or fermentative production by microorganisms, without particular limitation. The aromatic hydroxycarboxylic acid can be used, for example, as a form without being taken out as a crystal or then taken out as a crystal once, in crystal formation in the present invention.

A solvent which dissolves the aromatic hydroxycarboxylic acid is not particularly limited as long as it can dissolve the aromatic hydroxycarboxylic acid, and water is preferably used.

A dissolution temperature of the aromatic hydroxycarboxylic acid is preferably 60° C. or more, more preferably 70° C. or more from the viewpoint of complete dissolution of the aromatic hydroxycarboxylic acid, and is preferably 100° C. or less, more preferably 90° C. or less from the viewpoint of stability of the aromatic hydroxycarboxylic acid.

A solution including the aromatic hydroxycarboxylic acid in the present invention is preferably an aqueous solution derived from a culture solution of a microorganism from the viewpoint of industrial productivity.

The microorganism may be any of a wild-type strain, a mutant strain, or a variant strain in which variation such as insertion, substitution or deletion of a base sequence occurs by various genetic manipulations, or one may be adopted to which an ability to produce the aromatic hydroxycarboxylic acid is imparted by known artificial modification.

Examples of a microorganism having an ability to produce the aromatic hydroxycarboxylic acid include microorganisms of the genus Escherichia, the genus Rhodococcus, the genus Acinetobacter, the genus Bradyrhizobium, the genus Corynebacterium, the genus Pseudomonas, the genus Rhodopseudomonas, the genus Sinorhizobium, the genus Brevibacterium, the genus Novosphingobium, the genus Ralstonia, the genus Nocardioidaceae, the genus Microbacterium, the genus Streptomyces, the genus Amycolatopsis, the genus Kineococcus, the genus Pantoea, the genus Klebsiella, the genus Arthrobacter, and the like. Examples of a microorganism having an ability to produce gallic acid and protocatechuic acid preferably include microorganisms of the genus Escherichia, the genus Rhodococcus, the genus Acinetobacter, the genus Bradyrhizobium, the genus Corynebacterium, the genus Pseudomonas, the genus Rhodopseudomonas, the genus Sinorhizobium, the genus Brevibacterium, the genus Novosphingobium, the genus Ralstonia, and the like.

A culture medium used for culturing the microorganism having an ability to produce the aromatic hydroxycarboxylic acid preferably includes a carbon source, an inorganic nitrogen source or an organic nitrogen source which can be assimilated as a culture raw material by the microorganism, and other required organic trace nutrient source.

Examples of a culture medium used for culturing gallic acid and protocatechuic acid-producing bacteria include a CGXII culture medium and a CGCF culture medium (WO 2014/007273).

Examples of the carbon source include sugar (glucose, sucrose, maltose, and the like), organic acids, dextran, soluble starch, and methanol.

Examples of the inorganic nitrogen source or the organic nitrogen source include ammonium salts, nitrates, various amino acids, corn steep liquor, tryptone, peptone, casein, yeast extract, meat extract, soybean meal, and potato extract liquids.

There may also be included inorganic salts (sodium chloride, calcium chloride, sodium dihydrogen phosphate, potassium dihydrogen phosphate, dipotassium hydrogen phosphate, magnesium chloride, magnesium sulfate, manganese sulfate, and the like), vitamins, antibiotics (tetracycline, neomycin, kanamycin, spectinomycin, erythromycin, and the like), and the like.

A general method can be applied to culture of such a microorganism as long as conditions are adopted which can allow the microorganism to proliferate and produce the aromatic hydroxycarboxylic acid.

For example, when gallic acid and protocatechuic acid-producing bacteria are cultured, an LB culture medium, a CGXII culture medium, or the like can be used in preliminary preculture or preculture and a CGCF culture medium or the like can be used in main culture.

A culture temperature is preferably 20° C. or more, more preferably 30° C. or more, and preferably 40° C. or less, more preferably 35° C. or less.

A culture solution during culture has preferably a pH of 4 or more, more preferably a pH of 5 or more, and preferably a pH of 8 or less, more preferably a pH of 7 or less.

An amount of inoculation of such a microorganism to the culture medium is preferably 0.01% (v/v) or more, more preferably 0.1% (v/v) or more, further more preferably 0.5% (v/v) or more, and preferably 30% or less, more preferably 20% (v/v) or less, further more preferably 15% (v/v) or less.

A culture period of such a microorganism can be appropriately set depending on proliferation of the microorganism, and is preferably 0.1 days or more, more preferably 0.2 days or more, further more preferably 0.3 days or more, and preferably 20 days or less, more preferably 10 days or less, further more preferably 8 days or less, with 24 hours a day.

A conventionally known tank can be appropriately adopted as a culture tank used for culture. Examples include an aeration stirring-type culture tank, a bubble tower-type culture tank and a fluidized bed culture tank, and culture may be performed in any of a batch type, a semi-batch type and a continuous type.

Such culture provides a microorganism culture solution including the aromatic hydroxycarboxylic acid. The culture solution includes impurities, microbial bacteria and an unused culture raw material contaminated, in addition to the aromatic hydroxycarboxylic acid, and thus an aqueous solution including the aromatic hydroxycarboxylic acid is obtained by performing a separation operation such as centrifugation, membrane separation, or adsorption separation.

### (Content of Aromatic Hydroxycarboxylic Acid in Solution)

A content of the aromatic hydroxycarboxylic acid in the solution is not limited as long as it is equal to or less than a saturated solubility of the aromatic hydroxycarboxylic acid, and preferably 1 mass % or more, more preferably 2 mass % or more, further more preferably 2.5 mass % or more from the viewpoint of productivity. The content is preferably 20 mass % or less, more preferably 15 mass % or less, further more preferably 12 mass % or less from the viewpoint of an enhancement in yield in a bacteria separation operation. The content of the aromatic hydroxycarboxylic acid in the solution is preferably from 1 to 20 mass %, more preferably from 2 to 15 mass %, further more preferably from 2.5 to 12 mass %.

### (Polymer Molecule)

The polymer molecule used in the present invention is at least one selected from the group consisting of an anionic polymer molecule, a non-ionic polymer molecule and a cationic polymer molecule. These polymer molecules are preferably water-soluble. These polymer molecules may be each used in the form of a salt.

Examples of the anionic polymer molecule include a polymer having an anionic group such as a carboxyl group, a sulfate group, a sulfonate group, a phosphate group or a boronate group. Specific examples of a natural polymer molecule include xanthane gum, Arabic gum, and alginic acid and polyglutamic acid or each any salt thereof. Specific examples of a synthetic polymer molecule include a polymer or a copolymer formed from a monomer such as (meth)acrylic acid, maleic acid, maleic anhydride, fumaric acid, itaconic acid, crotonic acid or vinyl sulfonic acid, and any salt thereof. Examples also include carboxyalkyl cellulose such as carboxymethyl cellulose or carboxyethyl cellulose, and a carboxyvinyl polymer. These may be used singly or in combinations of a plurality thereof.

Examples of such any salt include an alkali metal salt, an alkali earth metal salt, an ammonium salt, an alkyl- or alkenyl-ammonium salt having from 1 to 22 carbon atoms, an alkyl- or alkenyl-substituted pyridinium salt having from 1 to 22 carbon atoms, an alkanol ammonium salt having from 1 to 22 carbon atoms, and a basic amino acid salt. Preferred is an alkali metal salt, and more preferred is a sodium salt or a potassium salt.

The anionic polymer molecule is preferably poly(meth)acrylic acid, xanthane gum, carboxyalkyl cellulose or any salt thereof, more preferably poly(meth)acrylic acid or any salt thereof, or xanthane gum, more preferably polyacrylic acid or any salt thereof, from the viewpoint of the effect of an increase in crystal grain size.

Examples of the non-ionic polymer molecule include starch-based polymer molecules (for example, soluble starch and methyl starch), cellulose-based polymer molecules (for example, alkyl cellulose such as methyl cellulose and ethyl cellulose, and hydroxyalkyl cellulose such as hydroxyethyl cellulose and hydroxypropyl methyl cellulose), vinyl polymer molecules (for example, polyvinyl alcohol, polyvinylpyrrolidone, and polyvinyl methyl ether), polyalkylene glycol, and a polyoxyethylene-polyoxypropylene block copolymer.

The non-ionic polymer molecule is preferably hydroxyalkyl cellulose, a vinyl polymer molecule obtained by polymerizing a monomer having a vinyl group (except for acrylic acid, much the same is true on the following), polyalkylene glycol, or a polyoxyethylene-polyoxypropylene block copolymer, more preferably hydroxyethyl cellulose, polyvinylpyrrolidone, polyethylene glycol, or a polyoxyethylene-polyoxypropylene block copolymer, from the viewpoint of the effect of an increase in crystal grain size.

Examples of the cationic polymer molecule include cationic cellulose, cationic starch, cationic guar gum, polyethylenimine-based polymers, dicyandiamide-based polymer molecules, and allylamine-based polymers.

The cationic polymer molecule is preferably an allylamine-based polymer, more preferably polyallylamine, from the viewpoint of the effect of an increase in crystal grain size.

The polymer molecule in the present invention is preferably one or more selected from the group consisting of a polymeric compound having a polyvinyl backbone, a polymeric compound having a sugar backbone, and a polymeric compound having an ethyleneoxy chain, more preferably a polymeric compound having a polyvinyl backbone, from the viewpoint of the effect of an increase in crystal grain size. Specifically, the polymer molecule is preferably at least one selected from the group consisting of poly(meth)acrylic acid or any salt thereof, xanthane gum, hydroxyalkyl cellulose, a vinyl polymer molecule obtained by polymerizing a monomer having a vinyl group, polyalkylene glycol, a polyoxyethylene-polyoxypropylene block copolymer, and an allylamine-based polymer, more preferably at least one selected from the group consisting of polyacrylic acid or any salt thereof, polyvinylpyrrolidone, polyethylene glycol, and polyallylamine, further more preferably polyacrylic acid or any salt thereof.

### (Molecular Weight of Polymer Molecule)

A weight average molecular weight of the polymer molecule is preferably 1,000 or more, more preferably 2,000 or more, further more preferably 4,000 or more from the viewpoint of the effect of an increase in crystal grain size, and is preferably 2,000,000 or less, more preferably 1,000,000 or less, further more preferably 500,000 or less from the viewpoint of filterability, and a water content in a cake after filtration. The weight average molecular weight of the polymer molecule is preferably from 1,000 to 2,000,000, more preferably from 2,000 to 1,000,000, further more preferably from 4,000 to 500,000.

The weight average molecular weight of the polymer molecule can be measured by, for example, a gel permeation chromatography (GPC) method.

### (Content of Polymer Molecule in Solution)

A content of the polymer molecule in the solution is from 0.0008 to 16 mass %. The content of the polymer molecule in the solution is 0.0008 mass % or more, preferably 0.001 mass % or more, more preferably 0.002 mass % or more, further more preferably 0.01 mass % or more from the viewpoint of the effect of an increase in crystal grain size, and is 16 mass % or less, preferably 10 mass % or less, more preferably 5 mass % or less, further more preferably 1 mass % or less from the viewpoint of industrial productivity, cost, and liquid viscosity. The content of the polymer molecule in the solution is from 0.0008 to 16 mass %, preferably from 0.001 to 10 mass %, more preferably from 0.002 to 5 mass %, further more preferably from 0.01 to 1 mass %.

A mass ratio % of the content of the polymer molecule to the content of the aromatic hydroxycarboxylic acid in the solution in the present invention (g-polymer molecule/g-aromatic hydroxycarboxylic acid) is preferably 0.015% or more, more preferably 0.05% or more, further more preferably 0.1% or more from the viewpoint of the effect of an increase in crystal grain size, and is preferably 300% or less, more preferably 50% or less, further more preferably 10% or less from the viewpoint of industrial productivity, cost, liquid viscosity and purity. The mass ratio of the content of the polymer molecule to the content of the aromatic hydroxycarboxylic acid in the solution is preferably from 0.015 to 300%, more preferably from 0.05 to 50%, further more preferably from 0.1 to 10%.

### (Crystallization Apparatus)

The aromatic hydroxycarboxylic acid crystal may be precipitated under still standing conditions, or may be precipitated under stirring with a reaction tank having a stirring blade.

The stirring blade may have any shape, and is preferably a paddle blade, a turbine blade, a propeller blade, an anchor blade, a large-blade-diameter paddle blade, or a max blend blade particularly in order to improve mixing of the crystal.

A circumferential velocity of stirring is preferably 0.2 m/s or more, more preferably 0.3 m/s or more, further more preferably 0.5 m/s or more from the viewpoint of uniform crystallization of aromatic hydroxycarboxylic acid having a high dissolution rate, and is preferably 10 m/s or less, more preferably 5 m/s or less, further more preferably 3 m/s or less from the viewpoint of suppression of crushing of the aromatic hydroxycarboxylic acid crystal.

### (Method for Precipitating)

A method for precipitating the aromatic hydroxycarboxylic acid crystal is not particularly limited, and such precipitating can be performed by an operation of a precipitation method by cooling, a precipitation method by pH adjustment, a precipitation method by concentration, a precipitation method by reaction, or the like. These precipitation methods may be performed singly or in combinations of a plurality of these methods. The precipitation method by cooling is preferred from the viewpoint of generation of an aromatic hydroxycarboxylic acid crystal having a high dissolution rate.

### (Precipitation Method by Cooling)

The precipitation method by cooling can crystallize the aromatic hydroxycarboxylic acid by cooling the solution from a high temperature to a low temperature and thus increasing a concentration of the aromatic hydroxycarboxylic acid to a solubility or more.

A preferred temperature of the solution before cooling is the same as the dissolution temperature of the aromatic hydroxycarboxylic acid.

A cooling temperature is preferably 50° C. or less, more preferably 40° C. or less, further more preferably 30° C. or less, and preferably 0° C. or more, more preferably 5° C. or more, further more preferably 8° C. or more from the viewpoint of a rate of recovery of the aromatic hydroxycarboxylic acid. The cooling temperature is preferably from 0 to 50° C., more preferably from 5 to 40° C., further more preferably from 8 to 30° C.

A cooling rate in precipitation of the aromatic hydroxycarboxylic acid crystal by cooling (average cooling rate calculated from a time taken for reaching the cooling temperature from a temperature of the solution before cooling) is not particularly limited as long as it is a cooling rate which can be achieved in a real crystallization tank, and is preferably 0.01° C./min or more, more preferably 0.05° C./min or more, further more preferably 0.1° C./min or more from the viewpoint of a cycle time, and is preferably 10° C./min or less, more preferably 5° C./min or less, further more preferably 1° C./min or less from the viewpoint of a cooling ability of the crystallization tank.

### (Precipitation Method by pH Adjustment)

The precipitation method by pH adjustment can crystallize the aromatic hydroxycarboxylic acid by adding an acid and thus detaching the aromatic hydroxycarboxylic acid from an aromatic hydroxycarboxylic acid salt to increase the concentration of the aromatic hydroxycarboxylic acid to a solubility or more.

Any acid can be used for pH adjustment without any particular limitation as long as it is smaller in pKa than the aromatic hydroxycarboxylic acid, and is particularly preferably an inorganic acid. Examples of the inorganic acid include hydrochloric acid, nitric acid, sulfuric acid, and phosphoric acid. Preferred is sulfuric acid or hydrochloric acid.

A pH in precipitation performed is adjusted to 9.0 or less, preferably 8.0 or less, more preferably 7.0 or less, as a pH at the start of crystallization, from the viewpoint of a rate of recovery of the aromatic hydroxycarboxylic acid, and is adjusted to more preferably 6.0 or less, more preferably 5.0 or less, further more preferably 4.0 or less by acid addition. The pH is preferably adjusted to 0.5 or more, more preferably 1.0 or more, further more preferably 1.5 or more from the viewpoint of corrosivity of a reaction tank or the like.

### (Precipitation Method by Concentration)

The precipitation method by concentration can crystallize the aromatic hydroxycarboxylic acid by evaporating a solvent (for example, water) of the solution for concentration to increase the concentration of the aromatic hydroxycarboxylic acid to a solubility or more.

A temperature in evaporation is not particularly limited, and is preferably 100° C. or less, more preferably 90° C. or less, further more preferably 80° C. or less, and preferably 5° C. or more, more preferably 10° C. or more, further more preferably 20° C. or more.

Such evaporation may also be performed under reduced pressure.

### (Precipitation Method by Reaction)

The precipitation method by reaction can be appropriately set depending on the type of the aromatic hydroxycarboxylic acid. The precipitation method by reaction also includes neutralization. For example, hydroxybenzoic acid, aminohydroxybenzoic acid, gallic acid, or protocatechuic acid can be crystallized.

### (Collection of Aromatic Hydroxycarboxylic Acid Crystal)

The aromatic hydroxycarboxylic acid crystal can be collected by a solid-liquid separation operation such as centrifugation, filtration, or decantation. The aromatic hydroxycarboxylic acid crystal may be, if necessary, washed. Examples of a solvent used for washing include water, ethanol, acetone, and toluene.

### (Drying of Aromatic Hydroxycarboxylic Acid Crystal)

Drying of the aromatic hydroxycarboxylic acid crystal is not particularly limited as long as moisture can be removed. For example, a common dryer can be used, for example, a shelf dryer, a conical dryer, a paddle dryer, a Nauta mixer, a fluidized bed dryer, a vacuum stirring dryer, or a disc dryer. Preferred is a drying method with no high shear force applied for maintaining an aromatic hydroxycarboxylic acid crystal structure.

A drying temperature is preferably −50° C. or more, more preferably −30° C. or more, further more preferably −20° C. or more, and preferably 90° C. or less, more preferably 80° C. or less, further more preferably 70° C. or less. Such drying may be made under reduced pressure.

The aromatic hydroxycarboxylic acid crystal dried may also be, if necessary, treated, for example, sieved.

### [Aromatic Hydroxycarboxylic Acid Crystal]

The aromatic hydroxycarboxylic acid crystal is thus obtained.

The aromatic hydroxycarboxylic acid crystal obtained by the method of the present invention has a large crystal size. An average crystal short diameter of the aromatic hydroxycarboxylic acid crystal is, preferably 3.0 μm or more, more preferably 4.0 μm or more, further more preferably 5.0 μm or more.

A ratio of increase in average crystal short diameter of the aromatic hydroxycarboxylic acid crystal according to the present invention relative to average crystal short diameter in a crystal obtained by crystallization in the absence of the polymer molecule is preferably 1.1 or more, more preferably 1.4 or more, further more preferably 1.8 or more, further more preferably 2.0 or more.

Herein, a digital microscope (Nikon ECLIPSE80i manufactured by Nikon Corporation, or VHX-5,000 manufactured by Keyence Corporation) is used in crystal observation and image analysis software ImageJ (developed by National Institutes of Health, NIH) is used in a measurement for short diameter of crystal. The detail of such a measurement method is described in Examples.

The aromatic hydroxycarboxylic acid crystal in the present invention not only is directly used, but also is useful as an intermediate raw material for production of various derivatives.

With respect to the embodiments described above, the present invention further discloses the following method for producing an aromatic hydroxycarboxylic acid crystal.

<1> A method for producing an aromatic hydroxycarboxylic acid crystal, the method comprising a step of precipitating an aromatic hydroxycarboxylic acid crystal from a solution comprising aromatic hydroxycarboxylic acid and at least one polymer molecule selected from the group consisting of an anionic polymer molecule, a non-ionic polymer molecule and a cationic polymer molecule, wherein a content of the polymer molecule in the solution is 0.0008 mass % or more and 16 mass % or less.

<2> The method for producing an aromatic hydroxycarboxylic acid crystal according to <1>, wherein the aromatic hydroxycarboxylic acid is preferably aromatic hydroxycarboxylic acid having one carboxy group and from one to three hydroxy groups, more preferably salicylic acid, γ-resorcinol acid, hydroxybenzoic acid, aminohydroxybenzoic acid, gallic acid, protocatechuic acid, or a compound obtained by substituting at least some hydrogen on an aromatic ring of such aromatic hydroxycarboxylic acid, with a substituent, further more preferably hydroxybenzoic acid, aminohydroxybenzoic acid, gallic acid or protocatechuic acid, even more preferably gallic acid or protocatechuic acid.

<3> The method for producing an aromatic hydroxycarboxylic acid crystal according to <1> or <2>, wherein the solution is preferably prepared by adding the polymer molecule to a solution comprising aromatic hydroxycarboxylic acid.

<4> The method for producing an aromatic hydroxycarboxylic acid crystal according to <3>, wherein the solution comprising aromatic hydroxycarboxylic acid is water comprising aromatic hydroxycarboxylic acid at preferably 60° C. or more, more preferably 70° C. or more, and preferably 100° C. or less, more preferably 90° C. or less.

<5> The method for producing an aromatic hydroxycarboxylic acid crystal according to <3>, wherein the solution comprising aromatic hydroxycarboxylic acid is preferably an aqueous solution derived from a culture solution of a microorganism.

<6> The method for producing an aromatic hydroxycarboxylic acid crystal according to <5>, further comprising a step of culturing a microorganism having an ability to produce the aromatic hydroxycarboxylic acid at a culture temperature of preferably 20° C. or more, more preferably 30° C. or more, and preferably 40° C. or less, more preferably 35° C. or less.

<7> The method for producing an aromatic hydroxycarboxylic acid crystal according to <6>, wherein a culture solution during culture has preferably a pH of 4 or more, more preferably a pH of 5 or more, and preferably a pH of 8 or less, more preferably a pH of 7 or less.

<8> The method for producing an aromatic hydroxycarboxylic acid crystal according to <6> or <7>, wherein an amount of inoculation of the microorganism having an ability to produce the aromatic hydroxycarboxylic acid to a culture medium is preferably 0.01% (v/v) or more, more preferably 0.1% (v/v) or more, further more preferably 0.5% (v/v) or more, and preferably 30% or less, more preferably 20% (v/v) or less, further more preferably 15% (v/v) or less.

<9> The method for producing an aromatic hydroxycarboxylic acid crystal according to any of <6> to <8>, wherein a culture period of the microorganism having an ability to produce the aromatic hydroxycarboxylic acid is preferably 0.1 days or more, more preferably 0.2 days or more, further more preferably 0.3 days or more, and preferably 20 days or less, more preferably 10 days or less, further more preferably 8 days or less, with 24 hours a day.

<10> The method for producing an aromatic hydroxycarboxylic acid crystal according to any of <1> to <9>, wherein a content of the aromatic hydroxycarboxylic acid in the solution is preferably 1 mass % or more, more preferably 2 mass % or more, further more preferably 2.5 mass % or more, preferably 20 mass % or less, more preferably 15 mass % or less, further more preferably 12 mass % or less, and preferably from 1 to 20 mass %, more preferably from 2 to 15 mass %, further more preferably from 2.5 to 12 mass %.

<11> The method for producing an aromatic hydroxycarboxylic acid crystal according to any of <1> to <10>, wherein the polymer molecule is preferably a water-soluble polymer molecule.

<12> The method for producing an aromatic hydroxycarboxylic acid crystal according to any of <1> to <11>, wherein the anionic polymer molecule is preferably a polymer having a carboxyl group, a sulfate group, a sulfonate group, a phosphate group or a boronate group, more preferably a natural polymer molecule (xanthane gum, Arabic gum, or alginic acid or polyglutamic acid or any salt thereof), a polymer or copolymer formed from a monomer ((meth)acrylic acid, maleic acid, maleic anhydride, fumaric acid, itaconic acid, crotonic acid or vinyl sulfonic acid), or any salt thereof, carboxyalkyl cellulose, or a carboxyvinyl polymer.

<13> The method for producing an aromatic hydroxycarboxylic acid crystal according to any of <1> to <12>, wherein the anionic polymer molecule is preferably poly(meth)acrylic acid, xanthane gum, carboxyalkyl cellulose or any salt thereof, more preferably poly(meth)acrylic acid or any salt thereof, or xanthane gum, more preferably polyacrylic acid or any salt thereof.

<14> The method for producing an aromatic hydroxycarboxylic acid crystal according to any of <1> to <13>, wherein the non-ionic polymer molecule is preferably hydroxyethyl cellulose, polyvinylpyrrolidone, polyethylene glycol, or a polyoxyethylene-polyoxypropylene block copolymer.

<15> The method for producing an aromatic hydroxycarboxylic acid crystal according to any of <1> to <14>, wherein the cationic polymer molecule is preferably cationic cellulose, cationic starch, cationic guar gum, a polyethylenimine-based polymer, a dicyandiamide-based polymer molecule, or an allylamine-based polymer, more preferably polyallylamine.

<16> The method for producing an aromatic hydroxycarboxylic acid crystal according to any of <1> to <10>, wherein the polymer molecule is preferably one or more selected from the group consisting of a polymeric compound having a polyvinyl backbone, a polymeric compound having a sugar backbone, and a polymeric compound having an ethyleneoxy chain, more preferably a polymeric compound having a polyvinyl backbone, further more preferably at least one selected from the group consisting of poly(meth)acrylic acid or any salt thereof, xanthane gum, hydroxyalkyl cellulose, a vinyl polymer molecule obtained by polymerizing a monomer having a vinyl group (except for acrylic acid), polyalkylene glycol, a polyoxyethylene-polyoxypropylene block copolymer, and an allylamine-based polymer, even more preferably at least one selected from the group consisting of polyacrylic acid or any salt thereof, polyvinylpyrrolidone, polyethylene glycol, and polyallylamine, even more preferably polyacrylic acid or any salt thereof.

<17> The method for producing an aromatic hydroxycarboxylic acid crystal according to any of <1> to <16>, wherein a weight average molecular weight of the polymer molecule is preferably 1,000 or more, more preferably 2,000 or more, further more preferably 4,000 or more, preferably 2,000,000 or less, more preferably 1,000,000 or less, further more preferably 500,000 or less, and preferably from 1,000 to 2,000,000, more preferably from 2,000 to 1,000,000, further more preferably from 4,000 to 500,000.

<18> The method for producing an aromatic hydroxycarboxylic acid crystal according to any of <1> to <17>, wherein a content of the polymer molecule in the solution is preferably 0.0008 mass % or more, more preferably 0.001 mass %, further more preferably 0.002 mass % or more, further more preferably 0.01 mass % or more, preferably 16 mass % or less, more preferably 10 mass % or less, further more preferably 5 mass % or less, further more preferably 1 mass % or less, and preferably from 0.0008 to 16 mass %, more preferably from 0.001 to 10 mass %, further more preferably from 0.002 to 5 mass %, further more preferably from 0.01 to 1 mass %.

<19> The method for producing an aromatic hydroxycarboxylic acid crystal according to any of <1> to <18>, wherein a mass ratio % of the content of the polymer molecule to the content of the aromatic hydroxycarboxylic acid in the solution (g-polymer molecule/g-aromatic hydroxycarboxylic acid) is preferably 0.015% or more, more preferably 0.05% or more, further more preferably 0.1% or more, preferably 300% or less, more preferably 50% or less, further more preferably 10% or less, and preferably from 0.015 to 300%, more preferably from 0.05 to 50%, further more preferably from 0.1 to 10%.

<20> The method for producing an aromatic hydroxycarboxylic acid crystal according to any of <1> to <19>, wherein the step of precipitating the crystal is preferably precipitation by cooling, and a cooling temperature is preferably 50° C. or less, more preferably 40° C. or less, further more preferably 30° C. or less, preferably 0° C. or more, more preferably 5° C. or more, further more preferably 8° C. or more, and preferably from 0 to 50° C., more preferably from 5 to 40° C., further more preferably from 8 to 30° C.

<21> The method for producing an aromatic hydroxycarboxylic acid crystal according to <20>, wherein a cooling rate is preferably 0.01° C./min or more, more preferably 0.05° C./min or more, further more preferably 0.1° C./min or more, and preferably 10° C./min or less, more preferably 5° C./min or less, further more preferably 1° C./min or less.

<22> The method for producing an aromatic hydroxycarboxylic acid crystal according to any of <1> to <19>, wherein the step of precipitating the crystal is preferably precipitation by pH adjustment, and a pH at the start of crystallization is preferably 9.0 or less, more preferably 8.0 or less, further more preferably 7.0 or less, further more preferably 6.0 or less, even more preferably 5.0 or less, even more preferably 4.0 or less, and preferably 0.5 or more, more preferably 1.0 or more, further more preferably 1.5 or more.

<23> The method for producing an aromatic hydroxycarboxylic acid crystal according to any of <1> to <19>, wherein the step of precipitating the crystal is preferably precipitation by concentration, and a temperature in evaporation is preferably 100° C. or less, more preferably 90° C. or less, further more preferably 80° C. or less, and preferably 5° C. or more, more preferably 10° C. or more, further more preferably 20° C. or more.

<24> The method for producing an aromatic hydroxycarboxylic acid crystal according to any of <1> to <19>, wherein the step of precipitating the crystal is preferably precipitation by reaction, more preferably precipitation by reaction comprising neutralization.

<25> The method for producing an aromatic hydroxycarboxylic acid crystal according to any of <1> to <24>, further comprising a step of drying the aromatic hydroxycarboxylic acid crystal, wherein a drying temperature is preferably −50° C. or more, more preferably −30° C. or more, further more preferably −20° C. or more, and preferably 90° C. or less, more preferably 80° C. or less, further more preferably 70° C. or less.

<26> The method for producing an aromatic hydroxycarboxylic acid crystal according to any of <1> to <25>, wherein an average crystal short diameter in the aromatic hydroxycarboxylic acid crystal is preferably 3.0 μm or more, more preferably 4.0 μm or more, further more preferably 5.0 μm or more.

<27> The method for producing an aromatic hydroxycarboxylic acid crystal according to any of <1> to <26>, wherein a ratio of increase in an average crystal short diameter in the aromatic hydroxycarboxylic acid crystal relative to an average crystal short diameter in a crystal obtained by crystallization in the absence of the polymer molecule is preferably 1.1 or more, more preferably 1.4 or more, further more preferably 1.8 or more, further more preferably 2.0 or more.

<28> The method for producing an aromatic hydroxycarboxylic acid crystal according to any of <1> to <27>, wherein the aromatic hydroxycarboxylic acid crystal is preferably a gallic acid or protocatechuic acid crystal.

### EXAMPLES

### [Method for Measuring Short Diameter of Crystal]

A crystal was microscopically observed using a digital microscope (Nikon ECLIPSE80i manufactured by Nikon Corporation, or VHX-5,000 manufactured by Keyence Corporation), and a short diameter of the crystal was measured using image analysis software (image analysis software ImageJ, developed by National Institutes of Health, NIH). The short diameter of at least 20 or more crystals in microscope images of several fields of view were measured, and an average value was calculated by arithmetic average.

A ratio of a short diameter of crystal based on an average short diameter in a crystal obtained by crystallization without addition of any additive was defined as a ratio of increase in short diameter of crystal.

### [Method for Measuring Liquid Content]

20 mL of a crystal slurry after crystallization was subjected to suction filtration by glass fiber filter paper (ADVANTEC GA-100 manufactured by Advantec Toyo Kaisha, Ltd.), thereby separating a solid and a liquid. A crystal recovered was placed on an aluminum dish (weight T) whose weight was measured in advance, and then a weight of the aluminum dish and a wet crystal (weight W1) was measured. Subsequently, the crystal was dried at 105° C. for 2 hours. After drying, a weight of the aluminum dish and a dry crystal (weight W2) was measured. A liquid content in a crystal precipitated was evaluated by calculation equation 1.

$$Liquid content ( \% ) = ( W 1 - W 2 ) ( g ) / ( W 2 - T ) ( g ) \times 100 \qquad ( Equation 1 )$$

### [Method for Measuring Concentration of Gallic Acid]

Gallic acid and protocatechuic acid in a culture solution were diluted 500-fold with an aqueous 35 mM sulfuric acid solution, and then quantitative determination was performed with liquid chromatography.

### (Analysis Conditions)

Analysis conditions of liquid chromatography were as follows: column: L-column ODS, eluent A: aqueous 0.1 M KH₂PO₄-0.1% (v/v) H₃PO₄ solution, eluent B: aqueous 70% (v/v) methanol solution, eluent switching: separation at from 5 to 20 min with a gradient from liquid A/liquid B=100/0 to 0/100, detector: DAD, detection wavelength: 210 nm, column temperature: 40° C., and amount of liquid injected: 5 μL.

[Method for Measuring pH]

A pH of an aqueous solution (undiluted solution) at 70° C. was measured using F-50 manufactured by Horiba Ltd.

### Comparative Example 1

A screw-top bottle having a volume of 50 mL was charged with 1.2 g of gallic acid monohydrate and 20 mL of distilled water, and then dissolution was conducted in a constant-temperature water tank at 75° C. in a tightly sealed state. Subsequently, the resulting solution was taken out from the constant-temperature water tank and then left to still stand at room temperature (25° C.) for 5 days, thereby precipitating a crystal. The crystal precipitated was collected by a spatula and then microscopically observed, and then a short diameter of crystal was measured. The short diameter of crystal was 2.7 μm. A liquid content was 63%.

### Comparative Example 2

A screw-top bottle having a volume of 50 mL was charged with 1.2 g of gallic acid monohydrate and 20 mL of distilled water, and then dissolution was conducted in a constant-temperature water tank at 75° C. in a tightly sealed state. Subsequently, malic acid was added to the aqueous solution so as to be 2.2% (g/g-gallic acid), and then dissolved at 75° C. The resulting solution was taken out from the constant-temperature water tank and then left to still stand at room temperature for 5 days, thereby precipitating a crystal. The crystal precipitated was collected by a spatula and then microscopically observed, and a short diameter of crystal was then measured to evaluate the ratio of increase in short diameter of crystal relative to Comparative Example 1. The results were as shown in Table 1.

### Example 1

A screw-top bottle having a volume of 50 mL was charged with 1.2 g of gallic acid monohydrate and 20 mL of distilled water, and then dissolution was conducted in a constant-temperature water tank at 75° C. in a tightly sealed state. Subsequently, an additive shown in Table 1 was added to the aqueous solution so as to be 2.2% (g/g-gallic acid), and then dissolved at 75° C. The resulting solution was taken out from the constant-temperature water tank and then left to still stand at room temperature for 5 days, thereby precipitating a crystal. The crystal precipitated was collected by a spatula and then microscopically observed, and a short diameter of crystal was then measured to evaluate the ratio of increase in short diameter of crystal relative to Comparative Example 1. The results were as shown in Table 1.

TABLE 1

| | | Ratio of | |
|---|---|---|---|
| | | increase | |
| | | in short | |
| | Classification | diameter | Liquid |
| Additive | of additive | of crystal | content |
| Not added (Comparative | — | 1 | 63% |
| Example 1) | | | |
| Malic acid (Comparative | — | 0.9 | — |
| Example 2) | | | |
| Xanthane gum | Anionic polymer | 2.0 | — |
| (manufactured by Tokyo | molecule | | |
| Chemical Industry Co., Ltd.) | | | |
| Hydroxyethyl cellulose | Nonionic | 1.6 | — |
| (from 200 to 300 mPa · s 2% in | polymer | | |
| Water at 20° C., manufactured | molecule | | |
| by Tokyo Chemical Industry | | | |
| Co., Ltd.) | | | |
| Polyoxyethylene- | Nonionic | 2.1 | — |
| polyoxypropylene block | polymer | | |
| copolymer | molecule | | |
| (Pluronic F127, weight | | | |
| average molecular weight 12 | | | |
| 600, manufactured by Sigma- | | | |
| Aldrich Co. LLC) | | | |
| Polyethylene glycol 6 000 | Nonionic | 9.0 | — |
| (weight average molecular | polymer | | |
| weight from 7 300 to 9 300, | molecule | | |
| manufactured by FUJIFILM | | | |
| Wako Pure Chemical | | | |
| Corporation) | | | |
| Polyvinylpyrrolidone | Nonionic | 38.6 | — |
| (Polyvinylpyrrolidone K30, | polymer | | |
| weight average molecular | molecule | | |
| weight 40 000, manufactured | | | |
| by FUJIFILM Wako Pure | | | |
| Chemical Corporation) | | | |
| Polyacrylic acid | Anionic polymer | 5.6 | 20% |
| (weight average molecular | molecule | | |
| weight 5 000, manufactured by | | | |
| FUJIFILM Wako Pure | | | |
| Chemical Corporation) | | | |
| Polyallylamine | Cationic | 2.5 | — |
| (Polyallylamine PAA-05, | polymer | | |
| weight average molecular | molecule | | |
| weight 5 000, manufactured by | | | |
| Nittobo Medical Co., Ltd.) | | | |

### Comparative Example 3

A screw-top bottle having a volume of 50 mL was charged with 1.2 g of gallic acid monohydrate and 20 mL of distilled water, and then dissolution was conducted in a constant-temperature water tank at 75° C. in a tightly sealed state. Subsequently, polyacrylic acid having a molecular weight of 5,000 was added to the aqueous solution so as to be 0.010% (g/g-gallic acid), and then dissolved at 75° C. The resulting solution was taken out from the constant-temperature water tank and then left to still stand at room temperature for from 2 to 5 days, thereby precipitating a crystal. The crystal precipitated was collected by a spatula and then microscopically observed, and a short diameter of crystal was then measured to evaluate the ratio of increase in short diameter of crystal relative to no additive system. The results were as shown in Table 2.

### Example 2

A crystal was precipitated in the same manner as in Comparative Example 3 except that polyacrylic acid having a different molecular weight, shown in Table 2, was added at a concentration shown in Table 2, to the aqueous solution. The crystal precipitated was collected by a spatula and then microscopically observed, and a short diameter of crystal was then measured to evaluate the ratio of increase in short diameter of crystal. The results were as shown in Table 2.

TABLE 2

| | | | | Ratio of |
|---|---|---|---|---|
| | Weight average | Additive | | increase in |
| | molecular | concentration | Amount added | short diameter of |
| Additive | weight | [mass % (w/v)] | [% (g/g-gallic acid)] | crystal |
| Not added | — | — | — | 1 |
| Polyacrylic acid | 5,000 | 0.00054% | 0.010% | 1.0 |
| (manufactured by FUJIFILM Wako Pure Chemical Corporation) | | | (Comparative | |
| | | | Example 3) | |
| Polyacrylic acid | 5,000 | 0.0011% | 0.018% | 2.8 |
| (manufactured by FUJIFILM Wako Pure Chemical Corporation) | | | | |
| Polyacrylic acid | 5,000 | 0.0031% | 0.052% | 4.9 |
| (manufactured by FUJIFILM Wako Pure Chemical Corporation) | | | | |
| Polyacrylic acid | 5,000 | 0.0056% | 0.093% | 2.7 |
| (manufactured by FUJIFILM Wako Pure Chemical Corporation) | | | | |
| Polyacrylic acid | 5,000 | 0.010% | 0.18% | 2.8 |
| (manufactured by FUJIFILM Wako Pure Chemical Corporation) | | | | |
| Polyacrylic acid | 5,000 | 0.030% | 0.55% | 2.3 |
| (manufactured by FUJIFILM Wako Pure Chemical Corporation) | | | | |
| Polyacrylic acid | 5,000 | 0.060% | 1.1% | 2.4 |
| (manufactured by FUJIFILM Wako Pure Chemical Corporation) | | | | |
| Polyacrylic acid | 5,000 | 0.24% | 4.4% | 4.7 |
| (manufactured by FUJIFILM Wako Pure Chemical Corporation) | | | | |
| Polyacrylic acid | 5,000 | 0.54% | 10% | 6.4 |
| (manufactured by FUJIFILM Wako Pure Chemical Corporation) | | | | |
| Polyacrylic acid | 5,000 | 1.1% | 20% | 4.9 |
| (manufactured by FUJIFILM Wako Pure Chemical Corporation) | | | | |
| Polyacrylic acid | 5,000 | 2.7% | 50% | 6.5 |
| (manufactured by FUJIFILM Wako Pure Chemical Corporation) | | | | |
| Polyacrylic acid | 5,000 | 10% | 184% | 5.2 |
| (manufactured by FUJIFILM Wako Pure Chemical Corporation) | | | | |
| Polyacrylic acid | 5,000 | 15% | 276% | 8.3 |
| (manufactured by FUJIFILM Wako Pure Chemical Corporation) | | | | |
| Polyacrylic acid | 25,000 | 0.12% | 2.2% | 5.8 |
| (manufactured by FUJIFILM Wako Pure Chemical Corporation) | | | | |
| Polyacrylic acid | 250,000 | 0.12% | 2.2% | 5.6 |
| (manufactured by FUJIFILM Wako Pure Chemical Corporation) | | | | |

### Comparative Example 4

A screw-top bottle having a volume of 50 mL was charged with 1.2 g of protocatechuic acid and 20 mL of distilled water, and then dissolution was conducted in a constant-temperature water tank at 75° C. in a tightly sealed state. Subsequently, the resulting solution was taken out from the constant-temperature water tank and then left to still stand at room temperature for 5 days, thereby precipitating a crystal. The crystal precipitated was collected by a spatula and then microscopically observed, and then a short diameter of crystal was measured.

### Example 3

A screw-top bottle having a volume of 50 mL was charged with 1.2 g of protocatechuic acid and 20 mL of distilled water, and then dissolution was conducted in a constant-temperature water tank at 75° C. in a tightly sealed state. Subsequently, polyacrylic acid (weight average molecular weight 5,000, manufactured by FUJIFILM Wako Pure Chemical Corporation) was added to the aqueous solution so as to be 2.2% (g/g-gallic acid), and then dissolved at 75° C. The resulting solution was taken out from the constant-temperature water tank and then left to still stand at room temperature for 5 days, thereby precipitating a crystal. The crystal precipitated was collected by a spatula and then microscopically observed, and a short diameter of crystal was then measured to evaluate the ratio of increase in short diameter of crystal relative to Comparative Example 4. The results were as shown in Table 3.

TABLE 3

| | | Ratio of increase |
|---|---|---|
| | Classification | in short diameter |
| Additive | of additive | of crystal |
| Not added (Comparative Example 4) | — | 1 |
| Polyacrylic acid | Anionic | 10.1 |
| (weight average molecular weight | polymer | |
| 5 000, manufactured by FUJIFILM | molecule | |
| Wako Pure Chemical Corporation) | | |

### Comparative Example 5

A screw-top bottle having a volume of 50 mL was charged with 1.2 g of gallic acid monohydrate and 20 mL of distilled water, and then dissolution was conducted in a constant-temperature water tank at 75° C. in a tightly sealed state. Subsequently, the resulting solution was taken out from the constant-temperature water tank, then placed in a reciprocating shaker, and then reciprocated and shaken at room temperature for 2 days (150 r/m), thereby precipitating a crystal. The crystal precipitated was collected by a spatula and microscopically observed to measure a short diameter of crystal.

### Example 4

A screw-top bottle having a volume of 50 mL was charged with 1.2 g of gallic acid monohydrate and 20 mL of distilled water, and then dissolution was conducted in a constant-temperature water tank at 75° C. in a tightly sealed state. Polyacrylic acid (weight average molecular weight 5,000, manufactured by FUJIFILM Wako Pure Chemical Corporation) was added to the aqueous solution so as to be 2.2% (g/g-gallic acid), and then dissolved at 75° C. The resulting solution was taken out from the constant-temperature water tank, then placed in a reciprocating shaker, and then reciprocated and shaken at room temperature for 2 days (150 r/m), thereby precipitating a crystal. The crystal precipitated was collected by a spatula and then microscopically observed, and a short diameter of crystal was then measured to evaluate the ratio of increase in short diameter of crystal relative to Comparative Example 5.

The results were as shown in Table 4.

TABLE 4

| | | Ratio of increase |
|---|---|---|
| | Classification | in short diameter |
| Additive | of additive | of crystal |
| Not added (Comparative Example 5) | — | 1 |
| Polyacrylic acid | Anionic | 2.4 |
| (weight average molecular weight | polymer | |
| 5 000, manufactured by FUJIFILM | molecule | |
| Wako Pure Chemical Corporation) | | |

### Comparative Example 6

### (Preparation of Fermentation Liquid)

A gallic acid-containing culture solution cultured at a pH of 6.5 with a transfectant of the genus Corynebacterium by a fermentation method was obtained. Sulfuric acid was added for adjustment to a pH of 4.0, and then the resultant was sterilized at 70° C. for one hour and thereafter separated using a filter press to bacteria and a solution, thereby recovering an aqueous gallic acid solution. Subsequently, the pH was adjusted to 2.6 with sulfuric acid, and then the resultant was allowed to pass through a 0.2-μm membrane filter to separate impurities. The concentration of gallic acid in the resulting aqueous solution was 70 g/L.

### (Crystallization Method)

20 mL of the resulting aqueous gallic acid solution was placed in a screw-top bottle having a volume of 50 mL, and then tightly sealed. The solution was left to still stand at room temperature for 5 days, thereby precipitating a crystal. The crystal precipitated was collected by a spatula and then microscopically observed, and then a short diameter of crystal was measured.

### Example 5

The same operation as in Comparative Example 6 was performed by just before the crystallization operation.

The resulting aqueous gallic acid solution was placed in a screw-top bottle having a volume of 50 mL, and then an additive shown in Table 5 was added to the aqueous solution so as to be 2.2% (g/g-gallic acid), and then dissolution was conducted in a constant-temperature water tank at 75° C. in a tightly sealed state. The resulting solution was taken out from the constant-temperature water tank and then left to still stand at room temperature for 5 days, thereby precipitating a crystal. The crystal precipitated was collected by a spatula and then microscopically observed, and a short diameter of crystal was then measured to evaluate the ratio of increase in short diameter of crystal relative to Comparative Example 6. The results were as shown in Table 5.

TABLE 5

| | | Ratio of |
|---|---|---|
| | | increase in |
| | Classification | short diameter |
| Additive | of additive | of crystal |
| Not added (Comparative Example 6) | — | 1 |
| Polyethylene glycol 6 000 | Nonionic polymer | 5.7 |
| (weight average molecular weight | molecule | |
| from 7 300 to 9 300, manufactured | | |
| by FUJIFILM Wako Pure Chemical | | |
| Corporation) | | |
| Polyvinylpyrrolidone | Nonionic polymer | 2.2 |
| (Polyvinylpyrrolidone K30, weight | molecule | |
| average molecular weight 40 000, | | |
| manufactured by FUJIFILM Wako | | |
| Pure Chemical Corporation) | | |
| Polyacrylic acid (weight average | Anionic polymer | 1.6 |
| molecular weight 5 000, | molecule | |
| manufactured by FUJIFILM | | |
| Wako Pure Chemical Corporation) | | |

### Comparative Example 7

A glass bottle having a volume of 19 mL was charged with 0.1 g of gallic acid monohydrate and 10 g of distilled water, and then dissolution was conducted at room temperature. Subsequently, the pressure was reduced to −0.095 MPaG in a decompression dryer and water was then evaporated over 16 hours for concentration, thereby precipitating a crystal. A crystal precipitated at the time of evaporation of 8 g of water was collected by a spatula and then microscopically observed, and a short diameter of crystal was then measured. The results were as shown in Table 6.

### Example 6

A glass bottle having a volume of 19 mL was charged with 0.1 g of gallic acid monohydrate and 10 g of distilled water, and then dissolution was conducted at room temperature. Subsequently, polyethylene glycol 6,000 (weight average molecular weight: from 7,300 to 9,300, manufactured by FUJIFILM Wako Pure Chemical Corporation) was added to the aqueous solution so as to be 2.2% (g/g-gallic acid), and then dissolved at room temperature. The pressure was reduced to −0.095 MPaG in a decompression dryer and water was then evaporated over 16 hours for concentration, thereby precipitating a crystal. A crystal precipitated at the time of evaporation of 8 g of water was collected by a spatula and then microscopically observed, and a short diameter of crystal was then measured to evaluate the ratio of increase in short diameter of crystal relative to Comparative Example 7. The results were as shown in Table 6.

TABLE 6

| | | Ratio of increase |
|---|---|---|
| | Classification | in short diameter |
| Additive | of additive | of crystal |
| Not added (Comparative Example 7) | — | 1 |
| Polyethylene glycol 6 000 (weight | Nonionic | 1.7 |
| average molecular weight from 7 300 | polymer | |
| to 9 300, manufactured by FUJIFILM | molecule | |
| Wako Pure Chemical Corporation) | | |

It found as shown in Table 1 to Table 6 that the aromatic hydroxycarboxylic acid crystal obtained by crystallization by the method of the present invention has an increased average crystal short diameter as compared with the average short diameter of a crystal obtained by crystallization in the absence of the polymer molecule. It found that the aromatic hydroxycarboxylic acid crystal according to the present invention, having a large average crystal short diameter, has a low liquid content and a high-purity aromatic hydroxycarboxylic acid crystal is obtained by the method of the present invention.

1: A method for producing an aromatic hydroxycarboxylic acid crystal, comprising:
precipitating an aromatic hydroxycarboxylic acid crystal from a solution comprising an aromatic hydroxycarboxylic acid and at least one polymer molecule selected from the group consisting of an anionic polymer molecule, a non-ionic polymer molecule, and a cationic polymer molecule;
wherein a content of the polymer molecule in the solution is 0.0008 to 16 mass %.

2: The method for producing an aromatic hydroxycarboxylic acid crystal according to claim 1, wherein a content of the aromatic hydroxycarboxylic acid in the solution is 1 to 20 mass %.

3: The method for producing an aromatic hydroxycarboxylic acid crystal according to claim 1, wherein a mass ratio % of the polymer molecule to the aromatic hydroxycarboxylic acid in the solution, g-polymer molecule/g-aromatic hydroxycarboxylic acid, is 0.015% to 300%.

4: The method for producing an aromatic hydroxycarboxylic acid crystal according to claim 1, wherein the polymer molecule is at least one selected from the group consisting of a polymeric compound having a polyvinyl backbone, a polymeric compound having a sugar backbone, and a polymeric compound having an ethyleneoxy chain.

5: The method for producing an aromatic hydroxycarboxylic acid crystal according to claim 1, wherein the polymer molecule is at least one selected from the group consisting of a poly(meth)acrylic acid or a salt thereof, a xanthane gum, a hydroxyalkyl cellulose, a vinyl-based polymer molecule obtained by polymerizing a monomer having a vinyl group other than acrylic acid, a polyalkylene glycol, a polyoxyethylene-polyoxypropylene block copolymer, and an allylamine-based polymer.

6: The method for producing an aromatic hydroxycarboxylic acid crystal according to claim 1, wherein a weight average molecular weight of the polymer molecule is 1,000 to 2,000,000.

7: The method for producing an aromatic hydroxycarboxylic acid crystal according to claim 1, wherein precipitating the crystal comprises precipitating by cooling.

8: The method for producing an aromatic hydroxycarboxylic acid crystal according to claim 7, wherein precipitating by cooling comprises cooling at a cooling rate of 0.01° C./min to 10° C./min.

9: The method for producing an aromatic hydroxycarboxylic acid crystal according to claim 1, wherein precipitating the crystal comprises precipitating by concentration.

10: The method for producing an aromatic hydroxycarboxylic acid crystal according to claim 1, wherein an average crystal short diameter of the aromatic hydroxycarboxylic acid crystal is 3.0 μm or more.

11: The method for producing an aromatic hydroxycarboxylic acid crystal according to claim 1, wherein the aromatic hydroxycarboxylic acid crystal is a crystal of gallic acid or protocatechuic acid.
